(19) United States
(12) Patent Application Publication
(10) Pub. No.: US 2017/0188980 A1
(43) Pub. Date: Jul. 6, 2017

(54) WEARABLE SENSOR BASED BODY MODELING

(71) Applicant: Empire Technology Development LLC, Wilmington, DE (US)

(72) Inventor: David Walter Ash, Bellevue, WA (US)

(73) Assignee: EMPIRE TECHNOLOGY DEVELOPMENT LLC, Wilmington, DE (US)

(21) Appl. No.: 14/988,771

(22) Filed: Jan. 6, 2016

(51) Int. Cl. A61B 5/00; A61B 90/98; A61B 5/103; A61B 5/11

(52) U.S. Cl. CPC A61B 5/744; A61B 5/0024; A61B 5/1127; A61B 5/1118; A61B 90/98; A61B 5/486; A61B 5/1036; A61B 2503/10; A61B 2505/09; A61B 2503/40; A61B 2562/0219

(57) ABSTRACT

Technologies are generally described to provide models of body based on information collected from sensors. In some examples, position information from wearable sensors attached to different portions of a body may be used to determine a posture and/or a position of one or more portions of the body. A three-dimensional (3D) model of the body may be generated as a 3D graph based on the based on the posture and/or position information and a deviation of the posture and/or the position of the portions of the body from an optimal posture and/or position may be determined. The 3D model may be generated as a three-regular graph, where vertices of the three-regular graph represent portions of the body augmented with the wearable sensors and edges of the three-regular graph represent portions of the body connected to each other.

### BACKGROUND

Unless otherwise indicated herein, the materials described in this section are not prior art to the claims in this application and are not admitted to be prior art by inclusion in this section.

A number of medical specialties and scientific disciplines are dedicated to the study of human and animal bodies under different circumstances. For example, the body's posture or position of different body portions while pert athletic activities or under physical therapy may be important to understanding effects of activities on the body. While recording position information and analyzing after the fact may provide useful information, such an approach may not provide real time data that may be useful for various purposes.

### SUMMARY

The present disclosure generally describes techniques to model human or animal bodies based on information collected from wearable sensors.

According to some examples, a system to model a body based on information received from multiple wearable sensors is described. An example system may include the multiple wearable sensors configured to capture position information associated with one or more portions of the body and a communication device configured to receive the captured position information from the multiple wearable sensors. The system may also include an analysts module that is configured to receive the captured position information from the communication device, analyze the captured position information to determine one or more of a posture and a position of the one or more portions of the body, and provide the determined one or more of the posture and the position to a consuming application.

According to other examples, a method to model a body based on information received from multiple wearable sensors is described. The method may include receiving position information associated with multiple portions of the body from the multiple wearable sensors; analyzing the received position information to determine one or more of a posture and a

position of the one or more portions of the body; generating a three-dimension& (3D) model of the body as a 3D graph; determining a deviation of the one or more of the posture and the position of the one or more portions of the body from an optimal one or more of the posture and the position of the one or more portions of the body; and providing the determined deviation to a consuming application.

According to further examples, an augmented reality (AR) based system to model a body based on information received from multiple wearable sensors is described. The system may include a communication device configured to receive captured position information from the multiple wearable sensors, a display device configured to display the corrective feedback in form of an AR scene, and an analysis module. The analysis module may be configured to analyze the received position information to determine one or more of a posture and a position of one or more portions of the body; generate a three-dimensional (3D) model of the body as a 3D graph; determine a deviation of the one or more of the posture and the position of the one or more portions of the body from an optimal one or more of the posture and the position of the one or more portions of the body; and determine a corrective feedback based on the deviation.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described, with additional specificity and detail through use of the accompanying drawings, in which:

FIG. 1 illustrates an example wearable sensor system implemented on a human body to model the human body;

FIG. 2 illustrates an example of capture of human body positions through wearable sensors, where the captured information may be used in an augmented reality (AR) device;

FIG. 3 illustrates an example system to capture human body positions through wearable sensors, analyze the captured information, and provide to consuming applications on various computing devices;

FIG. 4 illustrates examples of major components in a system for wearable sensor based body modeling;

FIG. 5 illustrates a general purpose computing device, which may be used to model human or animal bodies based on information collected from wearable sensors;

FIG. 6 is a flow diagram illustrating an example method to model human or animal bodies based on information collected from wearable sensors that may be performed by a computing device such as the computing device in FIG. 5; and

FIG. 7 illustrates a block diagram of an example computer program product, all arranged in accordance with at least sonic embodiments described herein.

### DETAILED DESCRIPTION

In the following detailed description, reference is made to the accompanying drawings, which form a part hereof In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to methods, apparatus, systems, devices, and/or computer program products related to modeling human or animal bodies based on information collected from wearable sensors,

Briefly stated, technologies are generally described to provide models of bodies based on information collected from sensors. In some examples, position information from wearable sensors attached to different portions of a body may be used to determine a posture and/or a position of one or more portions of the body. A three-dimensional (3D) model of the body may be generated as a 3D graph based on the based on the posture and/or position information, and a deviation of the posture and/or the position of the portions of the body from an optimal posture author position may be determined. The 3D model may be generated as a three-regular graph, where vertices of the three-regular graph represent portions of the body augmented with the wearable sensors and edges of the three regular graph represent portions of the body connected to, each other.

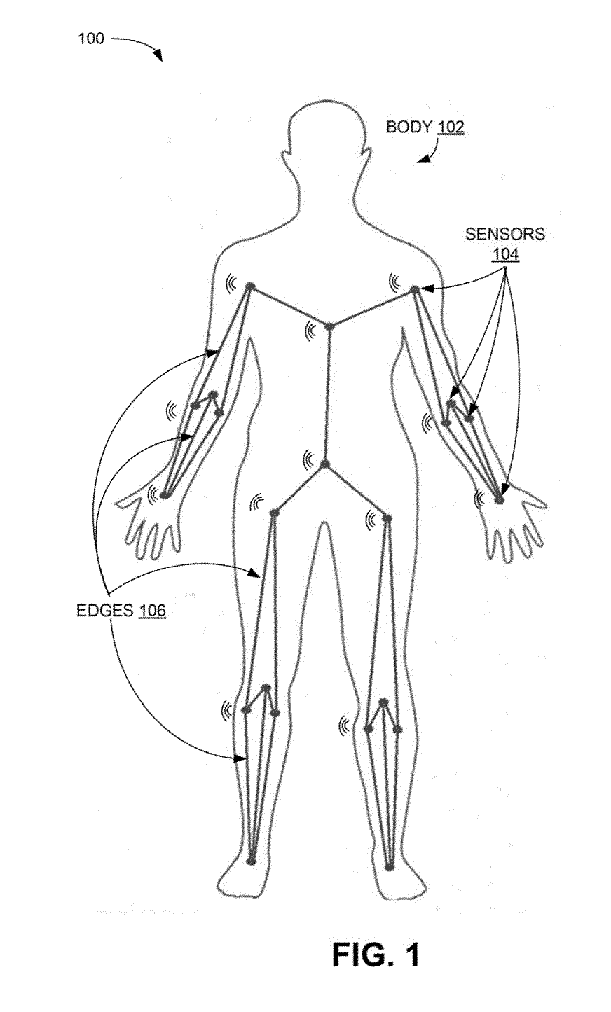

FIG. 1 illustrates an example wearable sensor system implemented on a human body to model the human body, arranged in accordance with at least some embodiments described herein.

As shown in a diagram 100, position information associated with various portions of a human body 102 may be obtained through multiple sensors 104 attached to different locations on the body 102. Real time information from the sensors 104 and analysis of body posture and/or position may provide information, for example, in sports activity environments (for example, potentially lifesaving information in sports such as BASE jumping) or in physical therapy environments, where activities may be adjusted based on the effects of the activity on the posture and position of various body parts. Furthermore, performance enhancement in sports may be achieved through real time feedback based on the information received from the sensors 104 and analysis based on a 3D model of the body.

The sensors 104 may include, but are not limited to, accelerometers, gyroscopic sensors, position sensors (e.g., rotational position), and/or plantar sensors. An optimal position of the body 102 may be previously established for an activity in question. For example, optimal positions may be available from databases based on testing of different populations, scientific modeling, or other sources. Based on the information obtained from the sensors 104, a discrepancy between the optimal posture and/or position of the body and the actual posture and/or position may be determined and feedback provided to the person performing the activity, another person overseeing the activity, etc. Thus, real time adjustment and corrections may be enabled through the feedback. Furthermore, presentation of the deviation on the 3D model of the body may provide a more realistic comparison of the effects.

According to some embodiments, the body may be modeled as a graph G=(V,E), where V may be an ordered set of vertices V={v₁, v₂, v₃, . . . vₖ} (for example, each vertex representing one of the sensors 104) and E may be an ordered set of edges 106 E={e₁, e₂, e₃, . . . , e₁}. Each edge may be an ordered pair of two vertices (representing connection between the two vertices), that is, eᵢ={vₗ, vᵣ}, where vₗ∈V and Vᵣ∈V. In another example, the vertices may represent parts of the body that are augmented with wearable sensors, and the edges may refer to two parts of the body such as a shoulder and an elbow, which are closely connected (i.e., with a connection of first degree between them). G may be a 3-regular graph. For some l, rₗ={v₁, v₂}. Any activity may be modeled as a mapping of body positions (based on vertices and edges) f :V× [symbol] → [symbol] ³, where each vertex represented by the body may optimally be at a certain point in 3D space at any given point in time. The actual position of the human body, which may or may not follow the optimal path may be presented as a similar mapping of body positions g:V× [symbol] → [symbol] ³. Thus, comparing f and g an analysis of the body's posture and/or position may be performed to determine a deviation from the optimal posture or position and provide corrective feedback.

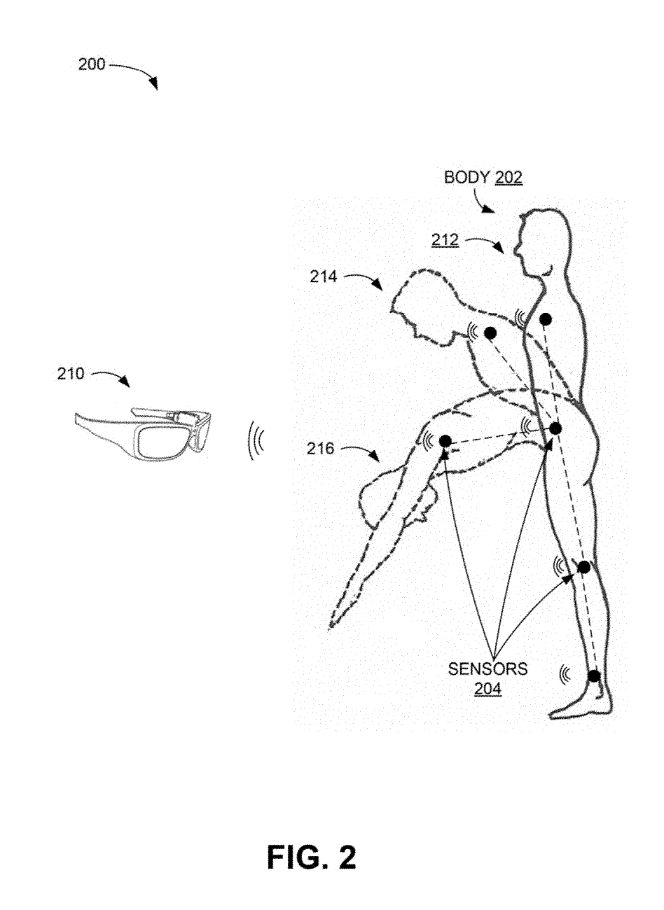

FIG. 2 illustrates an example of capture of human body positions through wearable sensors, where the captured information may be used in an augmented reality (AR) device, arranged in accordance with at least some embodiments described herein.

As shown in a diagram 200, a body 202 performing a sports activity may take different postures 212, 214, and 216 during the performance of that activity. Sensors 204 attached to different portions of the body 202 may detect position information, which may be used to determine the different postures at different times during the performance of the activity. In the illustrated example, the sensors 204 may allow positions of the torso and legs to be detected during performance of the activity. In other examples, the sensors may be placed at other locations allowing positions and/or postures of other body parts such as arms, feet, head, neck, etc. to be detected.

In some examples, the sensors 204 may form a small area network (a “body network”). The sensors 204 may be passive sensors, which may be interrogated by an active transponder (e.g., radio frequency identification (RFID) sensors) to retrieve the position information. The sensors 204 may also be active sensors and transmit the detected position information individually or through a designated correspondence sensor of the body network to a receiver via short-range transmission such as Bluetooth exchange. The information received (or retrieved) from the sensors 204 may be analyzed and processed at an analysis application being executed or executing on a computing device to determine the body posture and/or position. In yet other examples, the sensors may form a smart body network, where some or all of the processing may be performed centrally or in a distributed manner at the body network and the processed posture/position information may be transmitted to a consuming application. For example, a smart body suit may be designed with active and/or passive sensors, as well as one or more processors. The body suit may detect, analyze, and transmit posture/position information to other computing devices.

In the example configuration of the diagram 200, the sensors 204 may transmit the detected information to an AR application being executed on AR glasses 210, which may process the information and provide visual (and other) feedback to a user. The user may be the person performing the activity or another person monitoring the person performing the activity.

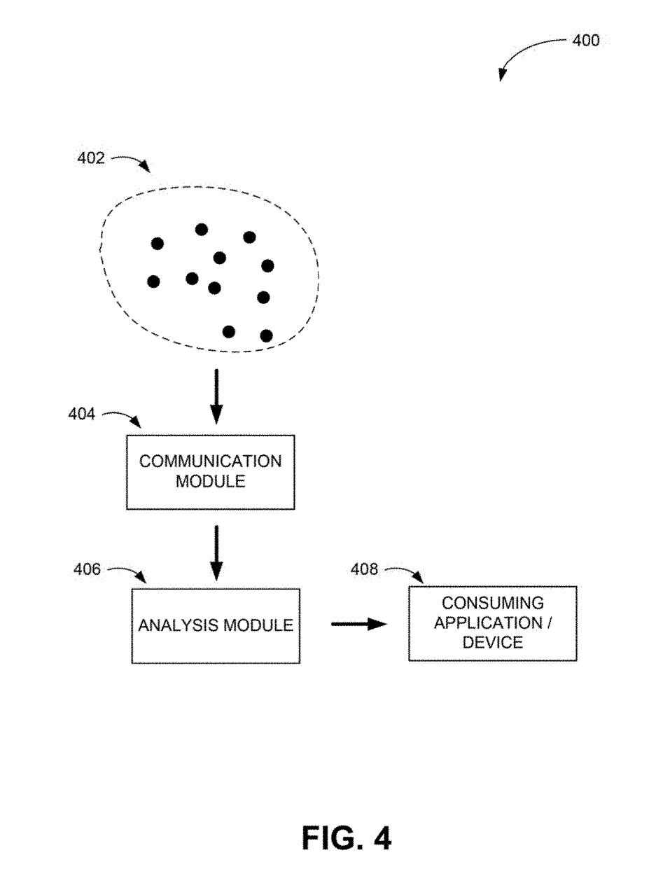

A regular graph is a graph where each vertex has the same number of neighbors, that is, every vertex has the same degree or valency. A regular graph with vertices of degree r is called an r-regular graph or regular graph of degree r. A 0-regular graph is made of disconnected vertices, a 1-regular graph is made of disconnected edges, and a 2-regular graph is made of disconnected cycles and infinite chains. 3-regular graph, also known as a cubic graph or 3-valent graph, is a graph in which all vertices have degree three. In some embodiments, the body may be modeled based on the information collected from the sensors 204 using a 3-regular graph approach. In other embodiments, other types of graphs such as distance-regular graphs or utility graphs may also be used. The modeling computation based on the received information is described in more detail below in conjunction with FIG. 4.

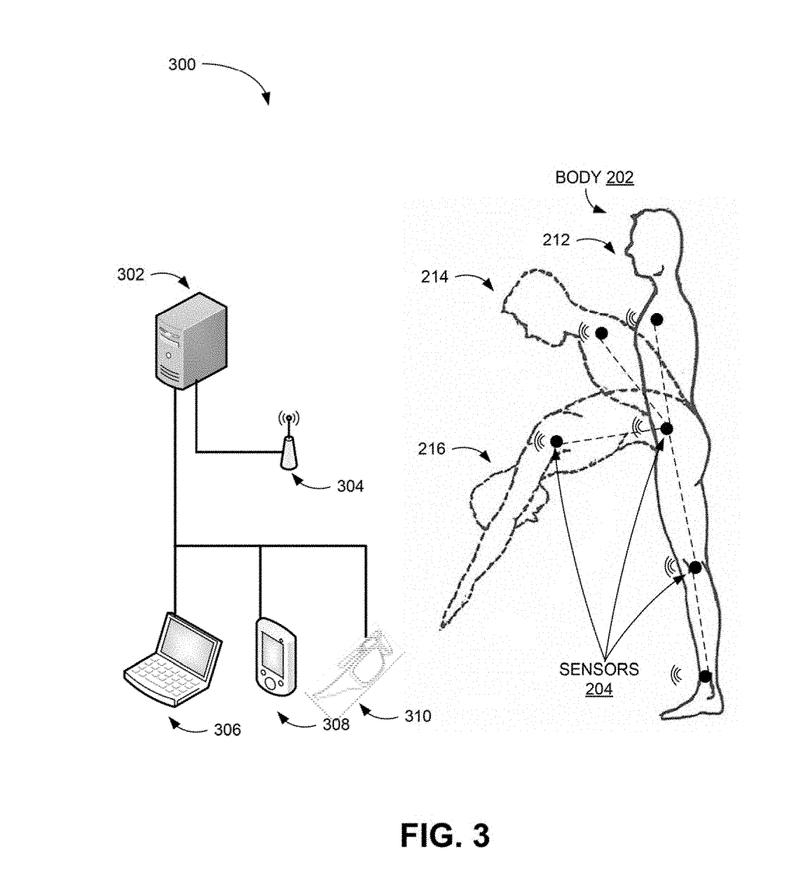

FIG. 3 illustrates an example system to capture human body positions through wearable sensors, analyze the captured information, and provide to consuming applications on various computing devices, arranged in accordance with at least some embodiments described herein.

As shown in a diagram 300, the postures 212, 214, and 216 of the body 202 may be determined based on position information provided by the sensors 204. The sensors 204 may transmit (actively or passively) the information to a variety of devices. In some examples, a single computing device such as a pair of AR glasses or a laptop computer may receive the information directly, process the information at or using an analysis application being executed on the computing device, and use the results to present the current body posture(s), deviations from optimal postures, or provide to other consuming applications fix- purposes such as further analysis, record keeping, enhanced presentations, and so on. In the illustrated configuration of the diagram 300, the information transmitted (wirelessly) by the sensors 204 may be received at a wireless receiver 304 communicatively coupled to a server 302. The server 302 may execute an analysis application and also store data associated with optimal postures for various activities and/or body types. The server 302 may provide results of the analysis or raw data to one or more computing devices such as laptop computer 306, handheld computer 308, and/or AR glasses 310.

In an example scenario, the analysis application executed at the server 302 may analyze a current posture for a particular body portion (e.g., legs), and compare that to an optimal posture for a particular activity being performed and body type (e.g., male, female, tall, short, heavy, thin, etc.). The result of the comparison may indicate a deviation from the optimal posture and/or a corrective feedback. The deviation and/or corrective feedback may be provided to the handheld computer 308 of the trainer and the AR glasses 310 worn by the person performing the activity.

While a human body is used in illustrative examples herein, animal bodies may be similarly modeled performing various activities. The applications and computing devices involved in the modeling and presentation of analysis results may also vary. Any application or group of applications, as well as computing devices may be used to provide corrective feedback to a user based on real time detection of body posture and/or position using the principles described herein. Furthermore, different communication technologies including, but not limited to, short range, long range, wired, wireless, optical, etc. may be used to exchange information between the sensors 204 and the various computing devices receiving the information.

FIG. 4 illustrates examples of major components in a system for wearable sensor based body modeling, arranged in accordance with at least some embodiments described herein.

As shown in a diagram 400, a group of sensors attached to a body may form a body network 402, which may collect position/posture information and provide the collected information to a communication module 404. The communication module 404 may provide the collected information to an analysis module 406, which may determine a time-based current body posture/position from the collected information, that is the body posture/position information for given time points. The time-based body posture/position information may be associated with a defined activity such as, sports activity or a physical therapy activity. The analysis module may also determine a deviation from a time-based optimal posture/position. The deviation may be determined based on a comparison of mapped locations of vertices e.g., sensors) and/or edges of the actual posture/position to the optimal posture/position. The analysis module 406 may provide the current posture/position information and/or the deviation information to a consuming application or device 408. The consuming application or device 408 may present the information to one or more users such as a person performing an activity, a trainer, students, referees, and/or other observers. The presentation may include, audible and or visual feedback.

The analysis module 406 or the consuming application or device 408 may model the body using a 3-regular graph approach. The modeling may implement following operations: First, E_{c} may be set to eₗ where eₗ is the edge satisfying eₗ={v₁, v₂} (see above), and V_{c} may be set to {v₁, v₂} may be selected, where eᵢ∉E_{c}, and vᵣ∉V_{c}. Thus, there would be three edges that meet at vₗ: eₗ_{₁},vₗ}, eₗ_{₂}={vᵣ_{₂}, vₗ}, and eₗ_{₃}, vₗ}. vᵣ_{₁} may be selected as vᵣ so that vᵣ_{₁}∉V_{c}. One can also make an assumption without loss of generality that vᵣ_{₃}∈V_{c}. In general, whether vᵣ_{₂}∈V_{c} or vᵣ_{₂}∉V_{c} may not be known.

Subsequently, for a given point in time t, f(vₗ, t)=(x,y,z), f(vᵣ_{ₗ},t)=(x₁y₁,z₁) f(vᵣ_{₂},t)=(x₂,y₂,z₂), and f (vᵣ_{₃},t)=(x₃y₃,z₃) may be supposed. The three angles θ₁, θ₂, and θ₃ for the edges may then be determined as follows:

$${θ}_{1} = {cos}^{- 1} \frac{( x - {x}_{2} ) ( x - {x}_{3} ) + ( y - {y}_{2} ) ( y - {y}_{3} ) + ( z - {z}_{2} ) ( z - {z}_{3} )}{\begin{matrix}\sqrt{{( x - {x}_{2} )}^{2} + {( y - {y}_{2} )}^{2} + {( z - {z}_{2} )}^{2}} * \\ \sqrt{{( x - {x}_{3} )}^{2} + {( y - {y}_{3} )}^{2} + {( z - {z}_{3} )}^{2}}\end{matrix}}$$

θ₂ and θ₃ may also be computed similarly. These three angles θ₁, θ₂, and θ₃ may represent the optimal angles at the vertex vₗ, which may correspond to a joint in the body, for example, at time t. Similar to the computation of θ₁, θ₂, and θ₃, angles ψ₁, ψ₂, and ψ₃ for the observed function g, may be computed representing the actual angles at the same joint at time t.

Having determined the optimal and actual angles for the joints, a tolerance threshold ε>0 may be set. If |θ₁-ψ₁|<ε,|θ₂-ψ₂|<ε, and |θ₃-ψε, then a recommendation may be made for no change at vertex vₗ as the body position there may be already adequate.

If the tolerance threshold is exceeded, however, two subcases may be considered. If vᵣ_{₂} ∈v_{c}, then a recommendation may be made for an adjustment to g(vᵣ_{₁},t) such that |θ₁-ψ₁∥θ₂-ψ₂∥θ₃-ψ₃| is minimized. And if vᵣ_{₂}∉V_{c}, then a recommendation may be made for an adjustment to g(vᵣ_{₁},t) and g(vᵣ_{₂},t) such that |θ₁-ψ₁∥θ₂-ψ₂∥θ₃-ψ₃| is minimized.

Next, V_{c}←V_{c}∪{vᵣ_{₁}, vᵣ_{₂}}and E_{c}←E_{c}∪{eₗ_{₁}, eₗ_{₂}} may be set. If V_{c}≠V, the computation may return to selection of eᵢ. When all vertices are covered, the computation may be terminated.

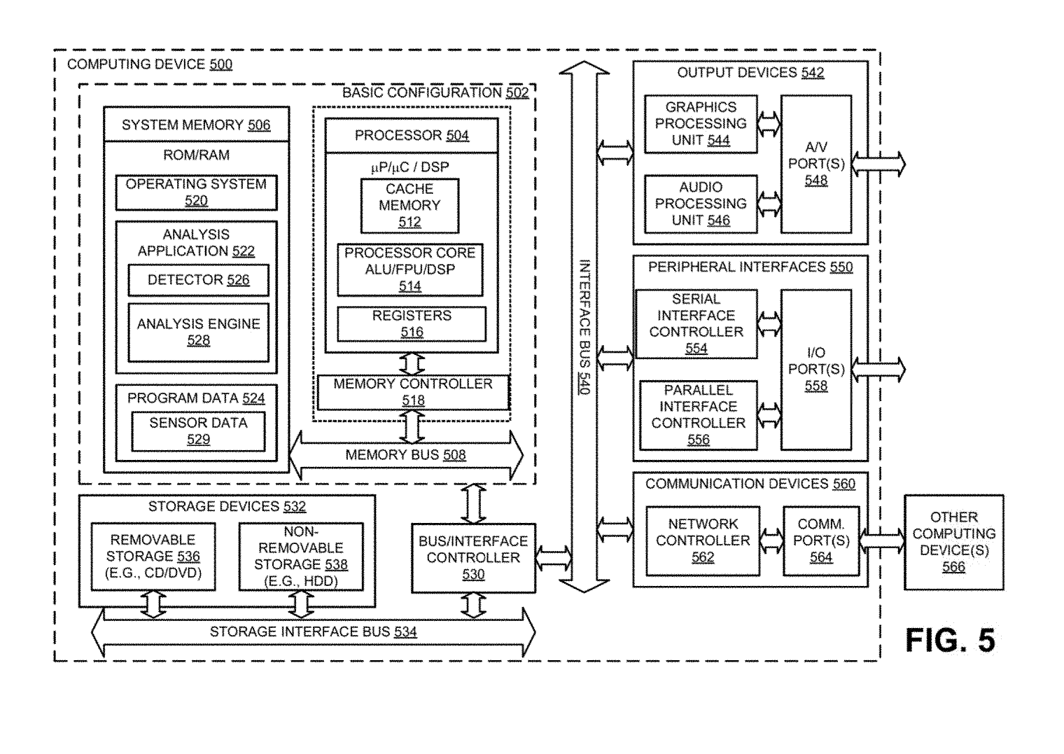

FIG. 5 illustrates a general purpose computing device, which may be used to provide user interface selection based on user context, arranged in accordance with at least some embodiments described herein.

For example, the computing device 500 may be used to select an appropriate user interface based, on user, context as described herein. In an example basic configuration 502, the computing device 500 may include one or more processors 504 and a system memory 506. A memory bus 508 may be used to communicate between the processor 504 and the system memory 506. The basic configuration 502 is illustrated in FIG. 5 by those components within the inner dashed line.

Depending on the desired configuration, the processor 504 may be of any type, including but not limited to a microprocessor (μP), a microcontroller (μC), a digital signal processor (Dsp), or any combination thereof. The processor 504 may include one more levels of caching, such as a cache memory 512, a processor core 514, and registers 516. The example processor core 514 may include an arithmetic logic unit (ALU), a floating point unit (FPU), a digital signal processing core (DSP Core), or any combination thereof. An example memory controller 518 may also be used with the processor 504, or in some implementations, the memory controller 518 may be an internal part of the processor 504.

Depending on the desired configuration, the system memory 506 may be of any type including but not limited to volatile memory (such as RAM), non-volatile memory (such as ROM, flash memory, etc.) or any combination thereof. The system memory 506 may include an operating system 520, an analysis application 522, and program data 524. The analysis application 522 may include a detector 526 configured to detect body position and status information from multiple sensors and an analysis engine 528 configured to determine a deviation of a posture and/or a position of one or more portions of the body from an optimal posture and/or the position of the portions of the body, as described herein. The program data 524 may include, among other data, sensor data 529 or the like, as described herein.

The computing device 500 may have additional features or functionality, and additional interfaces to facilitate communications between the basic configuration 502 and any desired devices and interfaces. For example, a bus/interface controller 530 may be used to facilitate communications between the basic configuration 502 and one or more data storage devices 532 via a storage interface bus 534. The data storage devices 532 may be one or more removable storage devices 536, one or more non-removable storage devices 538, or a combination thereof. Examples of the removable storage and the non-removable storage devices include magnetic disk devices such as flexible disk drives and hard-disk drives (HDD), optical disk drives such as compact disc (CD) drives or digital versatile disk (DVD) drives, solid state drives (SSDs), and tape drives to name a few. Example computer storage media may include volatile and nonvolatile, removable and non-removable media implemented in any method or technology for storage of information, such as computer readable instructions, data structures, program modules, or other data.

The system memory 506, the removable storage devices 536 and the non-removable storage devices 538 are examples of computer storage media. Computer storage media includes, but is not limited to, RAM, ROM, EEPROM, flash memory or other memory technology, CD-ROM, digital versatile disks (DVDs), solid state drives, or other optical storage, magnetic cassettes, magnetic tape, magnetic disk storage or other magnetic storage devices, or any other medium which may be used to store the desired information and which my be accessed by the computing device 500. Any such computer storage media may be part of the computing device 500.

The computing device 500 may also include an interface bus 540 for facilitating communication from various interface devices (e.g., one or more output devices 542, one or more peripheral interfaces 550, and one or more communication devices 560) to the basic configuration 502 via the bus/interface controller 530. Some of the example output devices 542 include a graphics processing unit 544 and an audio processing unit 546, which may be configured to communicate to various external devices such as a display or speakers via one or more A/V ports 548. One or more example peripheral interfaces 550 may include a serial interface controller 554 or a parallel interface controller 556, which may be configured to communicate with external devices such as input devices (e.g., keyboard, mouse, pen, voice Input device, touch input device, etc.) or other peripheral devices (e,g., printer, scanner, etc.) via one or more I/O ports 558. An example communication device 560 includes a network controller 562, which may be arranged to facilitate communications with one or more other computing devices 566 over a network communication link via one or more communication ports 564. The one or more other computing devices 566 may include servers at a datacenter, customer equipment, and comparable devices.

The network communication link may be one example of a communication media. Communication media may be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RE), microwave, infrared (IR) and other wireless media. The term computer readable media as used herein may include both storage media and communication media.

The computing device 500 may be implemented as a part of a general purpose or specialized server, mainframe, or similar computer that includes any of the above functions. The computing device 500 may also be implemented as a personal computer including both laptop computer and non-laptop computer configurations.

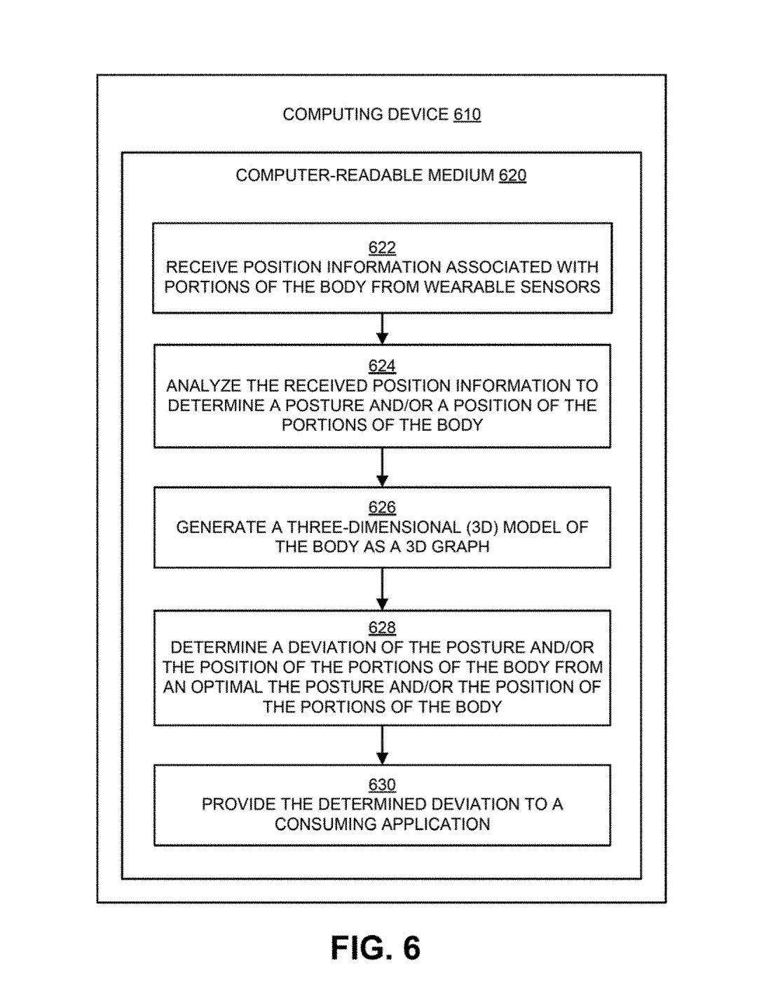

FIG. 6 is a flow diagram illustrating an example method to model human or animal bodies based on information collected from wearable sensors that may be performed by a computing device such as the computing device in FIG. 5, arranged in accordance with at least some embodiments described herein.

Example methods may include one or more operations, functions or actions as illustrated by one or more of blocks 622, 624, 626, 628, and/or 630, and may in some embodiments be performed by a computing device such as the computing device 610 in FIG. 6. The operations described in the blocks 622-630 may also be stored as computer-executable instructions in a computer-readable medium such as a computer-readable medium 620 of a computing device 610.

An example process to model human or animal bodies based on information collected from wearable sensors may begin with block 622, “RECEIVE POSITION INFORMATION ASSOCIATED WITH PORTIONS OF THE BODY FROM WEARABLE SENSORS”, where the body network 402 of sensors may detect position information and transmit actively or passively to a receiver (for example, an REID interrogator or a wireless receiver). The sensors may include accelerometers, gyroscopic sensors, plantar sensors, etc.

Block 622 may be followed by block 624, “ANALYZE THE RECEIVED POSITION INFORMATION TO DETERMINE A POSTURE AND/OR A POSITION OF THE PORTIONS OF THE BODY”, where the analysis module 406 may determine an actual posture or position of one or more body portions based on the information received from the sensors.

Block 624 may be followed by block 626, “GENERATE A THREE-DIMENSIONAL (3D) MODEL OF THE BODY AS A 3D GRAPH”, where the analysis module 406 or a consuming application 408 may generate a 3D model of the body using a 3-regular graph approach, where the vertices correspond to the sensors (or joints) and edges correspond to connections between the vertices. The 3D graph may be used to present the actual posture of the body or body portions to a user.

Block 626 may be followed by block 628, “DETERMINE A DEVIATION OF THE POSTURE AND/OR THE POSITION OF THE PORTIONS OF THE BODY FROM AN OPTIMAL THE POSTURE AND/OR THE POSITION OF THE PORTIONS OF THE BODY”, where the analysis module 406 or the consuming application 408 may compare the actual posture of the body to an optimal posture based on the 3D model and determine deviations. A preset threshold may be used to determine whether a corrective recommendation is needed or not.

Block 628 may be followed by block 630, “PROVIDE THE DETERMINED DEVIATION TO A CONSUMING APPLICATION”, where the deviation determined at block 678 and/or a corrective action recommendation may be provided to the consuming application 408 (if the determination is made by the analysis module 406). The consuming, application 408 may present the recommendation and/or current posture to one or more users including the person performing the activity (e.g., through AR glasses or perform other actions such as further analysis, record keeping, etc.

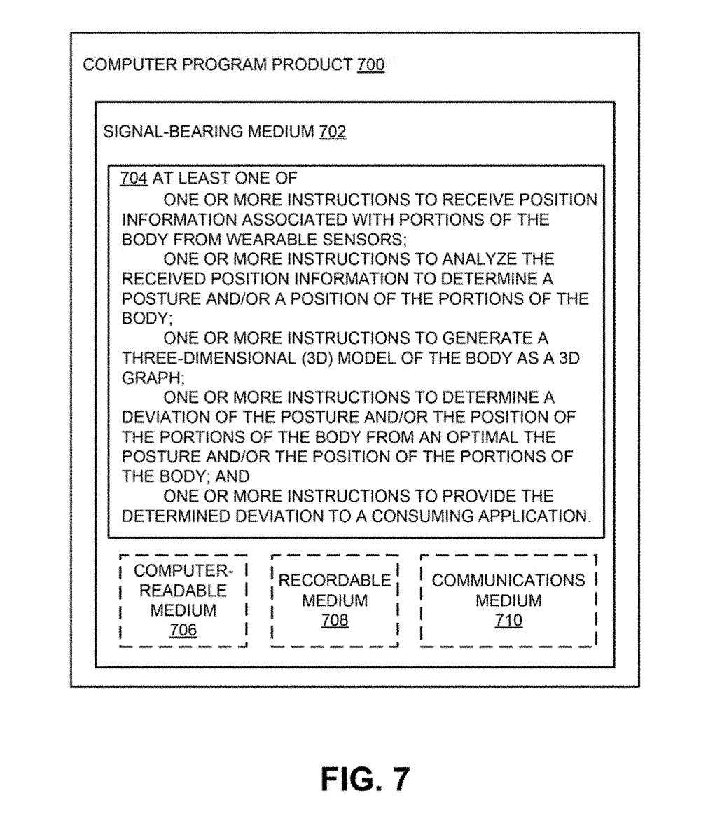

FIG. 7 illustrates a block diagram of an example computer program product, arranged in accordance with at least some embodiments described herein.

In some examples, as shown in FIG. 7, a computer program product 700 may include a signal,bearing medium 702 that may also include one or more machine readable instructions 704 that, when executed by, for example, a processor may provide the functionality described herein. Thus, for example, referring to the processor 504 in FIG. 5, the analysis application 522 may undertake one or more of the tasks shown in FIG. 7 in response to the instructions 704 conveyed to the processor 504 by the medium 702 to perform actions associated with modeling a body based on information received from a plurality of wearable sensors as described herein. Some of those instructions may include, for example, instructions to receive position information associated with a plurality of portions of the body from the plurality of wearable sensors; analyze the received position information to determine one or more of a posture and a position of the one or more portions of the body; generate a three-dimensional (3D) model of the body as a 3D graph; determine a deviation of the one or more of the posture and the position of the one or more portions of the body from an optimal one or more of the posture and the position of the one or more portions of the body; and provide the determined deviation to a consuming application, according to some embodiments described herein.

In some implementations, the signal bearing media 702 depicted in FIG. 7 may encompass computer-readable media 706, such as, but not limited to, a hard disk drive, a solid state drive, a compact disc (CD), a digital versatile disk (DVD), a digital tape, memory, etc. in some implementations, the signal bearing media 702 may encompass recordable media 708, such as, but not limited to, memory, read/write(R/W) CDs, R/W DVDs, etc. In some implementations, the signal bearing media 702 may encompass communications media 710, such as, but not limited to, a digital and/or an analog communication medium (e.g., a fiber optic cable, a waveguide, a wired communications link, a wireless communication link, etc.). Thus, for example, the program product 700 may be conveyed to one or more modules of the processor 504 by an RE signal bearing medium, where the signal bearing media 702 is conveyed by the wireless communications media 710 (e.g., a wireless communications medium conforming with the IEEE 802.11 standard).

According to some examples, a system to model a body based on information received from multiple wearable sensors is described. An example system may include the multiple wearable sensors configured to capture position information associated with one or more portions of the body and a communication device configured to receive the captured position information from the multiple wearable sensors. The system may also include an analysis module that is configured to receive the captured position information from the communication device, analyze the captured position information to determine one or more of a posture and a position of the one or more portions of the body, and provide the determined one or more of the posture and the position to a consuming application.

According to other examples, the system may further include a computing device configured to execute the consuming application, where the consuming application is configured to compare the determined one or more of the posture and the position to an optimal one or more of the posture and the position, and provide corrective feedback based on the comparison. The consuming application may be an augmented reality based application. The computing device may be a desktop computer, a handheld computer, a vehicle mount computer, or a wearable computer. The analysis module may be further configured to generate a three-dimensional (3D) model of the body as a graph comprising of. multiple vertices and edges, and determine a deviation of one or more of the vertices and edges from an optimal position.

According to further examples, the multiple vertices and edges may be an ordered set. The analysis module may also be configured to determine time-based positions of the multiple vertices and edges, and compare the time-based positions of the multiple vertices and edges to optimal time-based positions of the multiple vertices and edges. The time-based positions of the multiple vertices and edges may be categorized as a defined activity. The defined activity may be a sports activity or a physical therapy activity. The vertices may represent portions of the body augmented with the wearable sensors and the edges may represent portions of the body connected to each other. The communication device may be configured to receive the captured position information from the multiple wearable sensors through wireless communications. The multiple wearable sensors may include transmitters configured to transmit the captured position information upon an expiration of a predefined period or a request from the communication device.

According to other examples, a method to model a body based on information received from multiple wearable sensors is described. The method may include receiving position information associated with multiple portions of the body from the multiple wearable sensors; analyzing the received position information to determine one or more of a posture and a position of the one or more portions of the body; generating a three-dimensional (3D) model of the body as a 3D graph; determining a deviation of the one or, more of the posture and the position of the one or more portions of the body from an optimal one or more of the posture and the position of the one or more portions of the body; and providing the determined deviation to a consuming application.

According to yet other examples, generating the 3D model of the body as the 3D graph may include generating a three-regular graph, where vertices of the three-regular graph represent portions of the body augmented with the wearable sensors and edges of the three-regular graph represent portions of the body connected to each other. The method may also include determining an activity performed by the body by mapping locations of the multiple wearable sensors on the body in a time-based manner; retrieving a time-based map of body positions from a data source based on the determined activity; and/or determining the deviation by comparing the mapped locations of the multiple wearable sensors on the body to the time-based map of body positions. Receiving position information associated with the multiple portions of the body from the multiple wearable sensors data may include receiving the position information transmuted by the multiple wearable sensors. Receiving position information associated with the multiple portions of the body from the multiple wearable sensors data may also include interrogating radio frequency identification (RFID) tags embedded into the multiple wearable sensors.

According to further examples, an augmented reality (AR) based system to model a body based on information received from multiple wearable sensors is described. The system may include a communication device configured to receive captured position information from the multiple wearable sensors, a display device configured to display the corrective feedback in form of an AR scene, and an analysis module. The analysis module may be configured to analyze the received position information to determine one or more of a posture and a position of one or more portions of the body; generate a three-dimensional (3D) model of the body as a 3D graph; determine a deviation of the one or more of the posture and the position of the one or more portions of the body from an optimal one or more of the posture and the position of the one or more portions of the body; and determine a corrective feedback based on the deviation.

According to some examples, the analysis module may be further configured to determine time-based positions of multiple vertices and edges of the 3D graph; and compare the time-based positions of the multiple vertices and edges to optimal time-based positions of the multiple vertices and edges. The 3D graph may be a three-regular graph, the multiple vertices of the three-regular graph may represent portions of the body augmented with the wearable sensors and the multiple edges of the three-regular graph may represent portions of the body connected to each other. The body may be a human body or an animal body. The multiple wearable sensors may include one or more of plantar sensors, accelerometer sensors, and gyroscopic sensors.

There is little distinction left between hardware and software implementations of aspects of systems; the use of hardware or software is generally (but not always, in that in certain contexts the choice between hardware and software may become significant) a design choice representing cost vs. efficiency tradeoffs. There are various vehicles by which processes and/or systems and/or other technologies described herein may be effected (e.g., hardware, software, and/or firmware), and that the preferred vehicle will vary with the context in which the processes and/ systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples may be implemented, individually and/or collectively, by a wide range of hardware, software, firmware, or virtually any combination thereof. In one embodiment, several portions of the subject matter described herein may be implemented via application specific integrated circuits (ASICs), field programmable gate arrays (FPGAs), digital signal processors (DSPs), or other integrated formats. However, those skilled in the art will recognize that some aspects of the embodiments disclosed herein, in whole or in part, may be equivalently implemented in integrated circuits, as one or more computer programs executing on one or more computers (e.g., as one or more programs executing on one or more computer systems), as one or more programs executing on one or'more processors (e.g., as one or more programs executing on one or more microprocessors), as firmware, or as virtually any combination thereof, and that designing the circuitry and/or writing the code for the software and or firmware would be well within the skill of one of skill in the art in light of this disclosure.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is also to be understood that the terminology used herein is for the purpose of describing particular embodiments only, and is not intended to be limiting.

In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a compact disc (CD), a digital versatile disk (DVD), a digital tape, a computer memory, a solid state drive, etc.; and a transmission type medium such as a digital and or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein may be integrated into a data processing system via a reasonable amount of experimentation. Those having skill in the art will recognize that a data processing system may include one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity of gantry systems; control motors to move and/or adjust components and/or quantities).

A data processing system may be implemented utilizing any suitable commercially available components, such as those found in data computing/communication and/or network computing/communication systems. The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected”, or “operably coupled”, to each other to achieve the desired functionality, and any two components capable of being so associated may also be viewed as being “operably couplable”, to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect, to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “baying” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g.,” a system having at least one of A, B, and C” would include but not be limited to systems that have A alone, B alone, C alone, A and B together, A and C together, 13 and C together, and/or A, B, and C together, etc.). It will be further understood by those within the art. that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells, Similarly, a group having 1-5 cells refers to groups having 1. 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A system to model a body based on information received from a plurality of wearable sensors, the system comprising:
the plurality of wearable sensors configured to capture position information associated with one or more portions of the body;
a communication device configure(to receive the captured position information from the plurality of wearable sensors; and
an analysis module configured to:
receive the captured position information from the communication device;
analyze the captured position information to determine one or more of a posture and a position of the one or more portions of the body; and
provide the determined one or more of the posture and the position to a consuming application.

2. The system of claim 1, further comprising:
a computing device configured to execute the consuming application, wherein the consuming application is configured to:
compare the determined one or more of the posture and the position to an optional one or more of the posture and the position; and
provide corrective feedback based on the comparison.

3. The system of claim 2, wherein the consuming application is an augumented reality based application.

4. The system of claim 3, wherein the computing device is one of a desktop computer, a handheld computer, a vehicle mount computer, and a wearable computer.

5. The system of claim 1, wherein the analysis module is further configured to:
generate a three-dimensional (3D) model of the body as a graph comprising of a plurality of vertices and edges; and
determine a deviation of one or more of the plurality of vertices and edges from an optimal position,

6. The system of claim 5, wherein the plurality of vertices and edges are an ordered set.

7. The system of claim 5, wherein the analysis module is further configured to:
determine time-based positions of the plurality of vertices and edges; and
compare the time-based positions of the plurality of vertices and edges to optimal time-based positions of the plurality of vertices and edges.

8. The system of claim 7, wherein the time-based positions of the plurality of vertices and edges are categorized as a defined activity.

9. The system of claim 8, wherein the defined activity is one of a sports activity and a physical therapy activity.

10. The system of claim 5, wherein the vertices represent portions of the body augmented with the wearable sensors and the edges represent portions of the body connected to each other.

11. The system of claim 1, wherein the communication device is configured to receive the captured position information from the plurality of wearable sensors through wireless communications,

12. The system of claim 1, wherein the plurality of wearable sensors include transmitters configured to transmit the captured position information upon one of an expiration of a predefined period and a request from the communication device.

13. A method to model a body based on information received from a plurality of wearable sensors, the method comprising:
receiving position information associated with a plurality of portions of the body from the plurality of wearable sensors;
analyzing the received position information to determine one or more of a posture and a position of the One or more portions of the body;
generating a three-dimensional (3D) model of the body as a 3D graph;
determining a deviation of the one or more of the posture and the position of the one or more portions of the body from an optimal one or more of the posture and the position of the one or more portions of the body; and
providing the determined deviation to a consuming application.

14. The method of claim 13, wherein generating the 3D model of the body as the 3D graph comprises:
generating a three-regular graph, wherein vertices of the three-regular graph represent portions of the body augmented with the wearable sensors and edges of the three-regular graph represent portions of the body connected to each other.

15. The method of claim 14, further comprising:
determining an activity performed by the body by mapping locations of the plurality of wearable sensors on the body in a time-based manner.

16. The method of claim 15, further comprising:
retrieving a time-based map of body positions from a data source based on the determined activity; and
determining the deviation by comparing the mapped locations of the plurality of wearable sensors on the body to the time-based map of body positions.

17. The method of claim 13, wherein receiving position information associated with the plurality of portions of the body from the plurality of wearable sensors data comprises:
receiving the position information transmitted by the plurality of wearable sensors.

18. The method of claim 13, wherein receiving position information associated with the plurality of portions of the body from the plurality of wearable sensors data comprises:
interrogating radio frequency identification (RFID) tags embedded into the plurality of wearable sensors.

19. An augmented reality (AR) based system to model a body based on information received from a plurality of wearable sensors, the system comprising:
a communication device configured to receive captured position information from the plurality of wearable sensors;
an analysis module configured to:
analyze the received position information to determine one or more of a posture and a position of one or more portions of the body;
generate a three-dimensional (3D) model of the body as a 3D graph;
determine a deviation of the one or more of the posture and the position of the one or mom portions of the body from an optimal one or more of the posture and the position of the one or more portions of the body; and
determine a corrective feedback based on the deviation; and
a display device configured to:
display the corrective feedback in form of an R scene.

20. The system of claim 19, wherein the analysis module is further configured to:
determine time-based positions of a plurality of vertices and edges of the 3D graph; and
compare the time-based positions of the plurality of vertices and edges to optimal time-based positions of the plurality of vertices and edges.

21. The system of claim 20, wherein the 3D graph is a three-regular graph, the plurality of vertices of the three-regular graph represent portions of the body augmented with the wearable sensors and the plurality of edges of the three-regular graph represent portions of the body connected to each other.

22. The system of claim 19, wherein the body is one of a human body and an animal body.

23. The system of claim 19, wherein the plurality of wearable sensors include one or more of plantar sensors, accelerometer sensors, and gyroscopic sensors.
